(19) United States
(12) Patent Application Publication
(10) Pub. No.: US 2006/0258716 A1
(43) Pub. Date: Nov. 16, 2006

(54) Methods and compositions for the treatment of helicobacter pylori-associated diseases using endoperoxide bridge-containing compounds

(76) Inventors: Michael Marash, Rishon Lezion (IL); Elena Kluev, Jerusalem (IL)

Correspondence Address: FENNEMORE CRAIG, 3003 NORTH CENTRAL AVENUE, SUITE 2600, PHOENIX, AZ, 85012, US

(21) Appl. No.: 11/435,451

(22) Filed: May 16, 2006

Related U.S. Application Data
(63) Continuation of application No. PCT/IB04/03759, filed on Nov. 17, 2004.
(60) Provisional application No. 60/523,114, filed on Nov. 19, 2003.

(51) Int. Cl. A61K 31/7048; A61K 31/65; A61K 31/43; A61K 31/4439; A61K 31/4709; A61K 31/496; A61K 31/335

(52) U.S. Cl. 514/338; 514/452; 514/29; 514/192; 514/154; 514/253.08; 514/312; 514/400

(57) ABSTRACT

The present invention relates to methods and compositions for treating or preventing pathological conditions associated with ferrous-dependent bacteria, such as, Helicobacter pylori in which high intracellular ferrous iron concentration is required for survival and pathogenesis. The compositions of the invention comprise endoperoxide bridge-containing compounds that specifically inhibit the growth of the ferrous-dependent bacteria and preferably promote the eradication of the bacteria. The compositions, typically also include at least one active agent for treating Helicobacter sp-related gastrointestinal disorders, such as a proton pump inhibitor, an H2 blocker or a bismuth-containing compound.

### CROSS-REFERENCE TO RELATED APPLICATIONS

This application is a continuation of International Application PCT/IB2004/003759 filed Nov. 17, 2004, and which claims the benefit of U.S. provisional application 60/523,114 filed Nov. 19, 2003, the content of each of which is expressly incorporated herein by reference thereto.

### FIELD OF THE INVENTION

The present invention relates to methods and compositions for treating pathological conditions associated with ferrous-dependent bacteria, such as, Helicobacter pylori in which high intracellular ferrous iron concentration is required for survival and pathogenesis. The compositions of the invention comprise endoperoxide bridge-containing compounds that specifically inhibit the growth of the ferrous-dependent bacteria and preferably promote the eradication of the bacteria. The compositions typically also include at least one active agent for treating H. pylori-related gastrointestinal disorders, such as a proton pump inhibitor, an H2 blocker or a bismuth-containing compound.

### BACKGROUND OF THE INVENTION

Helicobacter pylori (H. pylori) is a gram-negative, microaerophilic bacterium which colonizes the human gastric mucosa for extended time periods. The infection, which concerns about half of the world population and stays lifelong if not treated, is the leading cause of ulcerations and a cofactor for the development of gastric adenocarcinoma and lymphoma.

H. pylori tolerates a wide range of local pH conditions and is relatively resistant to acid conditions. It is believed that this resistance is due in part to its production of urease that allows for the cleavage of urea, naturally present in gastric fluid and hence, the formation of a buffering ammonia layer surrounding the organism.

Proteins involved in iron metabolism are suggested to represent major virulence determinants of H. pylori. The dependence of H. pylori on iron uptake is disclosed for example in Velayudhan et al. (Molecular Microbiology, Vol. 37 p. 274, 2000). As revealed from this publication, ferrous iron uptake mediated by the transport protein FeoB is a prerequisite for the establishment of H. pylori infection in vivo. In contrast to other bacteria which use ferric iron as the main iron source, H. pylori is heavily dependent on ferrous iron, which is stabilized by the low pH and low oxygen concentration of the human stomach.

It is the combination of the unusual growth requirements and gastric location that makes the eradication of H. pylori so difficult. The ideal anti-microbial drug suitable for the successful treatment of H. pylori associated diseases should be stable at low pH values and should be able to readily penetrate the gastric mucosa. These desirable properties of an anti-microbial are not easily accomplished and thus, satisfactory treatment of H. pylori with anti-microbial drugs has yet to be accomplished.

Current antibiotic treatment for H. pylori infections usually consists of combinations of two antibiotic agents together with an adjunctive agent, which is usually either a Proton pump inhibitor (PPI) or H2 blocker. Antibiotic resistance of H. pylori is increasing in prevalence (Hazell, S L, Eur J Clin Infect Dis (1999) 18:83-86). Triple therapy regimen (Tetracycline, in combination with metronidazole and tripotassium dicitratobismuthate (TDB) or quadruple therapy in combination with a PPI has been found to be more effective than mono-therapy, but patient compliance and drug resistance further limits its applicability.

U.S. Pat. No. 5,196,205 (corresponding to international patent application WO 89/03219) describes a method for the treatment of H. pylori infections, consisting of the administration of a bismuth compound, an antibiotic belonging to the groups of penicillins and tetracycline, and a second antibiotic, such as metronidazole. The relevant therapy thus consists of the administration of three medications several times a day.

There are also other patents and patent applications describing single or multiple therapies for the eradication of H. pylori, such as U.S. Pat. Nos. 5,472,695, 5,560,912, 5,582,837, and international patent applications WO 92/11848 and WO 96/02237. None of these patents and patent applications overcomes the need to administer three medications several times a day.

Artemisinin is an anti-malarial drug isolated by Chinese scientists in 1972 from Artemisia annua L. The endoperoxide moiety of artemisinin and its analogs has been found to be necessary for the anti-malarial activity, and analogs lacking this group have been found to be inactive. In the presence of heme, the endoperoxide bridge undergoes reductive decomposition to form a free radical and electrophilic intermediates (Meshnick, Int. J. Parasitology, 32 (2002) 1655). It was recently proposed that artemisinin possesses its anti-parasite activity by inhibition of specific P-type ATPase (Eckstein-Ludwig et al., Nature, Vol. 424, 957).

Because of the low water solubility of the natural substance artemisinin, attempts have been made to convert it to a variety of synthetic derivatives in order to improve the pharmaceutical availability. Known analogs of artemisinin that have higher solubility in water are dihydroartemisinin, artemether, artesunate, arteether, propylcarbonate dihydroartemisinin and artelinic acid.

U.S. Pat. No. 4,978,676 discloses the use of artemisinin or artemisinin analogs in the treatment of skin conditions such as psoriasis, blistering skin diseases, viral warts, and hemorrhoids. U.S. Pat. No. 4,978,676 discloses the use of combinations of artemisinin and artemisinin analogs with monocarboxylic acids, esters or amides in the treatment of papulosquamous skin diseases, including psoriasis, an eczematous skin diseases, including seborrheic and atopic dermatitis. U.S. Pat. No. 5,219,880 discloses the use of artemisinin or artemisinin analogs in the treatment of warts, molluscum contagiosum and hemorrhoids. U.S. Pat. No. 5,225,427 discloses certain 10-substituted ether derivatives of dihydroartemisinin alleged to exhibit anti-malarial and anti-protozoal activity. Artemisinin has been shown to be toxic to cancer cells in vitro at 20-180 μM range (Sun et al., “Antitumor Activities of 4 Derivatives of Artemisic Acid and Artemisinin B in vitro,” Chung-Kuo-Yao-Li-Hsuch-Pao 13:541-543 (1992)). U.S. Pat. No. 5,578,637 discloses that the anticancer activity of compounds having an endoperoxide moiety such as artemisinin and its analogs, is substantially enhanced both in vitro and in vivo when administered under conditions which enhance intracellular iron concentrations.

WO04071506 discloses the use of Artemisinin and/or artemisinin derivatives for treating tumors induced by oncogenic viruses and for treating viral infections as well as treatment of cervical disorders associated with virus infection (e.g., cervical cancer and cervical dysplasia). This publication further discloses a method of killing or inhibiting growth of cells that are infected by oncogenic viruses such as BPV, HTLV-1, herpes virus (e.g., EBV or CMV), SV40-like viruses, hepatitis virus, or adenovirus.

WO04041176 discloses the use of sesquiterpene lactone endoperoxides to treat hepatitis C infections, yellow fever, dengue fever, bovine viral diarrhea and classical swine fever.

Foglio et al. disclose that dihydro-epideoxyarteannuin B and deoxyartemisinin provided gastric cytoprotection by decreasing the ulcerative lesion index produced by ethanol and indomethacin in rats (Planta Med. 2002, 68 515-518).

Few publications disclosed the use of artemisinin or artemisinin analogs as an anti-bacterial agent. U.S. Pat. No. 6,127,405 disclosed that α-arteether inhibits the growth of E. coli strains defective in DNA-gyrase enzyme whereas the wild type of E. coli having intact DNA gyrase genes were not sensitive to said α-arteethers. Shoeb et al. (J. Chemotherapy, 2, 362-367, 1990) disclosed that artemisinin possesses an anti-microbial activity against anaerobic bacteria. None of these publications disclose or suggest that artemisinin or its analogs may be used as an anti-bacterial agent against microaerophilic bacteria in general or specifically against bacteria which require high ferrous iron uptake for infection such as H. pylori.

There is a long felt need for an effective treatment for Helicobacter sp infections such as H. pylori infections, especially for H. pylori strains which are resistant to the presently marketed antibiotics.

### SUMMARY OF THE INVENTION

It is the object of the present invention to provide novel methods and compositions for inhibiting the growth of, or most preferably substantially eradicating, the ferrous-dependent bacteria, i.e., those bacteria that require high intracellular ferrous iron concentration for their survival and pathogenesis.

Another object of the present invention to provide novel methods for inhibiting the growth of, and most preferably substantially eradicating, ferrous-dependent bacteria within the gastric mucosa.

Yet another object of the present invention to provide novel methods for treating pathological conditions in the stomach associated with Helicobacter sp infections, preferably H. pylori infections.

A still further object of the present invention to provide novel methods and compositions for inhibiting the growth of H. pylori bacteria that are resistant to the antibiotics present in the art.

The present invention is directed in general to methods and compositions for the inhibition and most preferably the eradication of ferrous-dependent bacteria and pathogenesis associated therewith. The compositions of the present invention comprise a compound having an endoperoxide moiety that is reactive with the bacterial intracellular ferrous iron, which advantageously leads to the anti-bacterial effect.

While the compositions of the present invention are effective against any ferrous-dependent bacteria, preferred bacteria are bacteria residing within the gastric mucosa in which the high intracellular ferrous iron concentration is prerequisite for the establishment of infection in the acidic conditions of the stomach. The compositions of the present invention are especially effective against H. pylori bacteria that colonize the human gastric mucosa for extended time periods. Ferrous iron uptake mediated by the H. pylori transport protein FeoB is a prerequisite for the establishment of gastric H. pylori infection in vivo.

In one aspect, the present invention provides methods and compositions for inhibiting the growth of ferrous-dependent bacteria and for treating the pathogenesis associated therewith. The methods according to the present invention comprise administering to a subject in need thereof a growth inhibitory amount of a compound having an endoperoxide moiety that is reactive with ferrous iron present in high concentration in the bacteria. The methods of the present invention have been found to be especially effective against H. pylori bacteria that is an example of a ferrous-dependent bacteria, i.e., one that requires a high intracellular ferrous iron in order to colonize the human gastric mucosa for extended periods of time.

In another aspect, the present invention provides methods and compositions for treating pathological conditions associated with Helicobacter sp infections. The methods according to the present invention comprise administering to a subject in need thereof a growth inhibitory amount of a compound having an endoperoxide moiety that is reactive with ferrous iron present in high concentration in the bacteria. The methods of the present invention are especially effective against H. pylori bacteria that require high intracellular ferrous iron in order to colonize the human gastric mucosa for extended periods of time.

H. pylori is a microaerophilic gram-negative bacterium that is associated with multiple gastrointestinal pathologies, such as gastric peptic ulcer, duodenal peptic ulcer, gastritis, doudenitis, non-ulcer dyspepsia, gastric carcinoma and MALTOMA. Thus, the methods of the present invention may be used to prevent and treat gastrointestinal diseases or conditions associated with H. pylori.

In another aspect, the present invention provides methods for inhibiting the growth of antibiotic-resistant H. pylori strains in a subject in need thereof. The methods according to the present invention comprise administering to the subject a growth inhibitory amount of a compound having an endoperoxide moiety that is believed to react with ferrous iron present in high concentration in the bacteria to form toxic free radicals.

In a preferred embodiment, the endoperoxide-bearing compounds of the present invention have a sesquiterpene structure, particularly an oxygenated tricyclic sesquiterpene structure with an endoperoxide group, and preferably those which are sesquiterpene lactones or alcohols, carbonates, esters, ethers and sulfonates thereof. It will be apparent that other endoperoxide-bearing compounds may be useful for the present invention. Examples of other suitable endoperoxide-bearing compounds include for example: hydroxy, hydroperoxy or peroxy derivative of a polyunsaturated fatty acid, trioxolanes, spiro and dispiro 1,2,4-trioxolanes, byciclo (3,2,2) endoperoxides, trioxanes, 3-substituted trioxanes, ozonides, 2,3 bicyclo (3.3.1) nonanes, 1,2,4-trioxanes, 1,2,4,5-tetraoxanes, terpenes and substituted terpenes.

In a more preferred embodiment, the endoperoxide-bearing compound is a sesquiterpene compound, or a pharmaceutically acceptable salt thereof, according to formula (I):

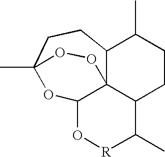

wherein R is —CO— or R is —CR₁—;

R₁ is hydrogen, hydroxyl, alkyl, —OR₂, —COR₂, —COR₂, —COOR₂, —CO(CH₂)ₙ, —COOH, or —SOOR₂;

wherein R₂ is alkyl or aryl; and n is 1 to 6.

As used herein, the term “alkyl” means lower alkyl having from 1 to 6 carbon atoms, preferably 1 to 4 carbon atoms. Alkyl groups of the invention may be straight-chain or branched-chain groups, with straight-chain groups preferred. The term “aryl” preferably refers to phenyl and benzyl, with phenyl the most preferred. Pharmaceutically acceptable salts include the alkali or alkaline metal salts, preferably sodium or potassium, with sodium being the most preferred.

Examples of such preferred compounds include artemisinin; dihydroartemisinin; carbonate, sulfonate, ester and ether derivatives of dihydroartemisinin, notably artemether, arteether, arteflene, artesunate, artesunate salts, dihydroartemisinin propyl carbonate, bis-ether artelinic acid and dihydroxydihydroartemisinin.

Advantageously, other compounds that possess an endoperoxide group that reacts in the presence of ferrous iron may be successfully used in the disclosed method, although in a non-limiting preferred embodiment the endoperoxide compounds are those disclosed herein

An example of one is a sesquiterpene compound, which includes, for example, artemisinin, where R of Formula (I) is C═O, dihydroartemisinin (R₁ is OH), artesunic acid (R₁ is OCO(CH₂)₂ CO₂H), and artesunate, artemether (R₁ is OCH₃) and arteether (R₁ is OC₂H₅).

An artemisinin molecule, as a representative endoperoxide compound of the present invention, is a sesquiterpene lactone containing an endoperoxide bridge that can be catalyzed by iron to form toxic free radicals. The present invention takes advantage of this property of artemisinin and targets it towards H. pylori bacteria. This selectivity in action is because ferrous iron uptake mediated by the H. pylori transport protein FeoB is a prerequisite for the establishment of H. pylori infection in vivo. The sesquiterpene compounds of the present invention possess an endoperoxide bridge structure. Peroxides generate toxic free radicals in a Fenton-type reaction when exposed to unbound ferrous iron. Thus, the increased ferrous iron concentration inside the bacteria may lead to intracellular free radical formation in the presence of the sesquiterpene compounds and cell death. It is also possible that artemisinin possesses its anti-bacterial ferrous-dependent activity via a different mechanism as suggested for example by Eckstein-Ludwig et al (Nature, Vol. 424, 957).

In addition to the endoperoxide-containing compounds, the compositions of the present invention may further comprise one or more active agents for treating H. pylori-related gastrointestinal pathologies as a means of further enhancing clinical efficacy. Such agents are for example an inhibitor of gastric acid secretion, a proton pump inhibitor (either irreversible or reversible proton pump inhibitor), an H2-blocker, bismuth salts, an antibiotic agent, an anti-inflammatory agent to treat the inflammation in the mucosa associated with H. pylori infection, a cytoprotectant such as sucralfate, prostaglandin analogues such as misoprostol, or iron in order to increase intracellular iron concentration.

The compositions of the present invention are specifically useful for eradicating H. pylori in the stomach. In one embodiment, artemisinin or the active derivatives thereof are formulated in a composition designed to act locally in the stomach following oral administration. Since artemisinin or the active derivatives thereof are not soluble in the acidic conditions of the gastric fluid, it is necessary to preserve its solubility in the stomach in order to permit the active compound to act locally.

Thus, the compositions may further comprise an agent that maintains the solubility of the endoperoxide-bearing compound in the gastric fluids. This enables the endoperoxide-bearing compound to act locally in the stomach against the bacteria. Such agents are preferably alkaline agents or antacids that when dissolved in the gastric juice are capable of elevating the pH of the gastric fluids to a pH in which at least significant proportion of the endoperoxide-bearing compound remains soluble in the gastric fluids.

According to various embodiments, the compositions may further comprise one or more agents that improve the availability of the endoperoxide-bearing compound to the bacteria within the gastric mucosa. Such agents are for example mucolytic agents that reduce the viscosity of the gastric mucosa, thereby accelerating the ability of the endoperoxide-bearing compound to reach the bacteria and act locally in the stomach rather then via the systemic circulation.

In order to accelerate the local effect of artemisinin or the active derivatives thereof in the stomach it is recommended to extend its gastric retention time. Thus in another embodiment, the compositions may further comprise one or more gastric-retention agents. These gastric-retention agents enable the active compound to act locally in the stomach for extended time periods sufficient to eradicate the bacteria.

Such gastric retention agents may be for example one or more polymers that swell in the stomach via the absorption of water from the gastric fluid, thereby increasing the size of the particles to promote gastric retention in the stomach. The active ingredient is slowly released from the particles by diffusion or following slow erosion of the particles in the stomach.

According to another embodiment, the compositions of the present invention are formulated to permit systemic absorption of the endoperoxide-bearing compound in the intestine. In order to accelerate the absorption of the endoperoxide-bearing compound in the intestine, the compositions may comprise vehicle such as vegetable oil suitable for liquid formulations that increase the absorption in the intestine.

The compositions of the present invention may be administered by intravenous, parenteral, or oral means. Although any suitable route of administration is acceptable according to the present invention, it is preferred to administer the compositions orally. The active compounds are typically combined with a pharmaceutically acceptable carrier to form a pharmaceutical composition. The pharmaceutically acceptable carrier can contain a physiologically acceptable compound that acts, for example, to stabilize the composition or to increase the absorption of the agent.

The compositions may further comprise one or more agents that accelerate the solubility and the stability of the endoperoxide-bearing compound in aqueous environment. Such agents are for example cyclodextrin analogs that form complexes with artemisinin or its derivatives, thereby improving the aqueous solubility of the complex and the stability of the endoperoxide bridge in aqueous environment.

In a preferred embodiment, the invention is a pharmaceutical composition for inhibiting the growth of a ferrous-dependent bacterial strain. In this embodiment, the composition preferably comprises

(a) a pharmaceutically effective amount of a compound according to formula (I):

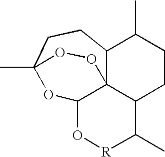

wherein R is —CO— or R is —CR₁—;

R₁ is hydrogen, hydroxyl, alkyl, —OR₂, —COR₂, —COR₂, —COOR₂, —CO(CH₂)ₙ, —COOH, or —SOOR₂;

R₂ is alkyl or aryl; and n is 1 to 6′ and

(b) one or more active agents for treating H. pylori-related gastrointestinal pathologies such as an antibiotic agent, an inhibitor of gastric acid secretion, a proton pump inhibitor (PPI), a reversible proton pump inhibitor, an H2 blocker, a bismuth-containing compound a cytoprotectant, prostaglandin analogues such as misoprostol or an anti-inflammatory agent. Most, preferably the pharmaceutical composition is designed to be most effective against a strain of a Helicobacter sp, such as Helicobacter pylori.

The pharmaceutical composition preferable, in general, comprises an amount of each active component, namely the endoperoxide-containing compound and the active agent for treating H. pylori-related gastrointestinal pathologies, sufficient to inhibit the growth of the bacteria if administered alone. In a preferred embodiment, the ratio of the endoperoxide-containing compound verses the active agent for treating H. pylori-related gastrointestinal pathologies is from about 50:1 to about 1:100 and more preferably 10:1 to 1:50. In another preferred embodiment, the endoperoxide-containing compound is artemisinin or artesunate and the active agent for treating H. pylori-related gastrointestinal pathologies is PPI.

In one specific embodiment, the present invention relates to novel oral formulations comprising an endoperoxide-containing compound, preferable a sesquiterpene and more preferable an artemisinin or an active derivative thereof and a PPI. Advantageously, the oral compositions may further comprise an antibiotic. Such oral dosage forms may contain one or both of the drugs in immediate or sustained release form such as in a gastric retention form.

The oral dosage forms may be in the form of tablets, capsules, troches, lozenges, aqueous or oily suspensions, dispersible powders or granules, emulsions, multiparticulate formulations, syrups, elixirs, and the like.

According to one embodiment, the oral compositions comprising artemisinin or an active derivative thereof and PPI in a single oral dosage form, preferably double-layered tablets or hard gelatin capsules. The combined oral composition may further comprise an antibiotic.

According to another embodiment, the oral compositions comprising artemisinin or an active derivative thereof, the PPI and possibly an antibiotic are in a separate oral dosage form such as tablets or capsules. According to various embodiments of the present invention, the PPI may be administered in enteric-coated form or non-enteric-coated form.

These and further embodiments will be apparent from the detailed description and examples that follow.

### BRIEF DESCRIPTION OF THE FIGURES

FIG. 1 shows the Minimal Inhibitory Concentration values of Artemisinin for E. coli (E.c), P. acnes (P.a), Lactobacillus acidophilus (L.a) and H. pylori (H.p), demonstrating the specificity of artemisinin against H. pylori.

FIG. 2 shows that Clarithromycin and metronidazole-resistant strains of H. pylori are sensitive to artemisinin.

FIG. 3 shows that artemisinin and omeprazole exhibit a synergistic effect on the eradication of H. pylori.

FIG. 4 shows the Minimal Inhibitory Concentration values of Artemisinin and active derivatives thereof for H. pylori.

FIG. 5 demonstrates that prolonged exposure of artesunate to H. pylori bacterial cultures results in irreversible bacterial eradication.

FIG. 6 demonstrates that Artesunate preserves its anti-Helicobacter activity even after prolonged incubation in low pH conditions.

FIG. 7 demonstrates that Artesunate efficiently reduces the number of colony forming units in H. pylori-infected mice treated with artesunate versus placebo.

### DETAILED DESCRIPTION OF THE INVENTION

The present invention relates generally to methods and compositions for inhibiting the growth bacteria that require high intracellular ferrous iron for their survival, i.e., ferrous-dependent bacteria, and pathogenesis, for example, Helicobacter species, such as H. pylori. In a preferred embodiment the method and composition are designed so as to substantially eradicate the ferrous-dependent bacteria. The term “substantially eradicate” preferably means that at least 50%, more preferably 75%, and most preferably at least 95% of the ferrous-dependent bacteria are killed.

The compositions of the present invention comprise a compound having an endoperoxide moiety that is believed to react with the bacterial intracellular ferrous iron, leading to the anti-bacterial effect.

In a preferred embodiment, the present invention relates to a composition comprising an endoperoxide-containing sesquiterpene compound, such as, for example, artemisinin or an active derivative thereof. These combinations are efficient and selective at inhibiting growth of ferrous-dependent bacteria, such as, H. pylori and other Helicobacter species, in the stomach while retaining the normal flora of the intestine intact. Furthermore, the composition of the present invention is useful for the inhibition or more preferably substantial eradication of H. pylori strains which are resistant to the conventional antibiotics.

Artemisinin, a preferred sesquiterpene compound used in the present invention has been shown to work through oxygen and carbon based free radical mechanisms. Its structure includes an endoperoxide bridge. Peroxides generate free radicals in a Fenton type reaction when exposed to unbound ferrous iron. It is possible that in the presence of artemisinin, high ferrous iron concentration inside the bacteria leads to intracellular free radical formation and cell death. Ferrous iron catalyzes the production of toxic hydroxyl radicals from hydrogen peroxide, which arises from the spontaneous combination of superoxide anions created by oxidative metabolism in cells. Hydroxyl radicals are highly destructive, damaging lipids, proteins and nucleic acids in the cell. Radicals induce the formation of unsaturated bonds in lipids, decreasing membrane fluidity and causing cell lysis. They also react with thiol groups in proteins, causing cross-linking and inactivation. Hydroxyl radicals can also extract hydrogen atoms from DNA and RNA, causing mutations or cleavage of the phosphodiester backbone.

It is also possible that artemisinin possess its anti-bacterial ferrous-dependent activity via a different mechanism as suggested for example by Eckstein-Ludwig et al (Nature, Vol. 424, 957). Eckstein-Ludwig et al., have suggested that artemisinin possesses its anti-parasite activity by inhibition of specific P-type ATPase. The anti-parasitic activity of artemisinin requires the presence of ferrous iron as the chelation of iron abrogates the anti-parasitic activity.

The compositions of the present invention are specifically useful for eradicating H. pylori in the stomach. The compositions of the present invention may further include as optional ingredients one or more agents already known for their use in the therapy of H. pylori-associated gastrointestinal pathologies, for added clinical efficacy. Preferred agents to be administered in combination with artemisinin or artemisinin derivatives are a proton pump inhibitor (PPI), an H2-blocker, bismuth salts, or an antibiotic effective against H. pylori. The oral compositions may further comprise iron in order to increase the intracellular iron concentration within the bacteria, so that the effectivity of the endoperoxide-containing molecules to inhibition of the bacterial growth is increased.

Numerous proton pump inhibitors are known to those of skill in the art. Thus, for example, U.S. Pat. No. 6,093,738 describes novel thiadiazole compounds that are effective as proton pump inhibitors. European Patent Nos. 322133 and 404322 disclose quinazoline derivatives, European Patent No. 259174 describes quinoline derivatives, and WO 91/13337 and U.S. Pat. No. 5,750,531 disclose pyrimidine derivatives, as proton pump inhibitors. Suitable proton pump inhibitors are also disclosed for example in EP-A1-174726, EP-A1-166287, GB 2 163 747 and WO90/06925, WO91/19711, WO91/19712, WO94/27988 and WO95/01977, all incorporated herein, in their entirety. In general, any proton pump inhibitor that is activated within the acid canaliculi and inhibits the activity of the H⁺/K⁺-adenosine triphosphatase (ATPase) proton pump may be used in combination with the endoperoxide-containing compound of the present invention. Particularly preferred PPIs include, but are not limited to omeprazole, esomeprazole, rabeprazole, lansoprazole, tenatoprazole and pantoprazole and derivatives or analogues thereof.

The oral compositions may further comprise an antibiotic for the treatment of ulcers associated with Helicobacter sp infection (e.g. Helicobacter pylori). Such antibiotics include, for example, amoxicillin, clarithromycin or other macrolides, metronidazole and related antibiotics, tetracycline, quinolones, rifabutin or furazolidone.

The PPIs used in the present invention can be used in neutral form or in the form of a salt (e.g., an alkaline salt), such as for instance the Mg⁺², CA⁺², NA⁺, K⁺, or Li⁺ salts, preferably the Mg⁺² salts. Further where applicable, the compounds can be used in racemic form or in the form of an enantiomer thereof, or salts of the racemates or the single enantiomers.

The active compounds used in the methods of the present invention may be administered by intravenous, parenteral, or oral means. In preferred embodiments of the present invention, the pharmaceutical compositions are administered orally. Such oral dosage forms may contain the active compound in immediate or sustained release form.

The compositions may further comprise one or more agents that improve the availability of the endoperoxide-bearing compound to the bacteria within the gastric mucosa, thereby permitting local activity of the endoperoxide-bearing compound against the bacteria. Such agents are for example mucolytic agents that reduce the viscosity of the gastric mucosa, thereby accelerating the ability of the endoperoxide-bearing compound to reach the bacteria. Such mucolytic agents are for example reducing agents such as N-acetyl cysteine, dithiothreitol, citric acid or mannitol.

Additionally, the compositions may further comprise an agent that maintains the solubility of the endoperoxide-bearing compound in the gastric fluids. This enables the endoperoxide-bearing compound to act locally in the stomach against the bacteria. Such agents are preferably alkaline agents or antacids that when dissolved in the gastric juice are capable of elevating the pH of the gastric fluids to a pH in which at least significant proportion of the endoperoxide-bearing compound remains soluble in the gastric fluids.

Alkaline agents to be used in the present invention include for example: sodium or potassium bicarbonate, magnesium oxide, hydroxide or carbonate, magnesium lactate, magnesium glucomate, aluminum hydroxide, aluminum, calcium, sodium or potassium carbonate, phosphate or citrate, di-sodium carbonate, disodium hydrogen phosphate, a mixture of aluminum glycinate and a buffer, calcium hydroxide, calcium lactate, calcium carbonate, calcium bicarbonate, and other calcium salts. It is noted that while sodium bicarbonate dissolves easily in water, calcium carbonate is water-insoluble and is slowly soluble only in acidic environment. Therefore, calcium carbonate may be useful when sustained dissolution of the alkaline agent in the stomach is desired.

Examples of antacids to be used in the present invention include one or more of the following: alumina, calcium carbonate, and sodium bicarbonate; alumina and magnesia; alumina, magnesia, calcium carbonate, and simethicone; alumina, magnesia, and magnesium carbonate; alumina, magnesia, magnesium carbonate, and simethicone; alumina, magnesia, and simethicone; alumina, magnesium alginate, and magnesium carbonate; alumina and magnesium carbonate; alumina, magnesium carbonate, and simethicone; alumina, magnesium carbonate, and sodium bicarbonate; alumina and magnesium trisilicate; alumina, magnesium trisilicate, and sodium bicarbonate; alumina and simethicone; alumina and sodium bicarbonate; aluminum carbonate, basic; aluminum carbonate, basic, and simethicone; aluminum hydroxide; calcium carbonate; calcium carbonate and magnesia; calcium carbonate, magnesia, and simethicone; calcium carbonate and simethicone; calcium and magnesium carbonates; magaldrate; magaldrate and simethicone; magnesium carbonate and sodium bicarbonate; magnesium hydroxide; magnesium oxide.

In order to accelerate the local effect of the endoperoxide-bearing compound in the stomach it is recommended to extend its gastric retention time. Thus in another embodiment, the compositions may further comprise one or more gastric-retention agents. These gastric-retention agents enable the active compound to act locally in the stomach for extended time periods sufficient to eradicate the bacteria.

Such gastric retention agents may be for example one or more polymers that swell in the stomach via the absorption of water from the gastric fluid, thereby increasing the size of the particles to promote gastric retention in the stomach. The active ingredient is slowly released from the particles by diffusion or following slow erosion of the particles in the stomach.

Polymers suitable for use as gastric retention agents have the property of swelling as a result of absorbing water from the gastric fluid, and gradually eroding over a time. The erosion properties of the polymer in the stomach resulting from the interaction of fluid with the surface of the dosage form are determined mainly by the polymer molecular weight and the drug/polymer ratio. In order to ensure a gradual erosion over few hours, it is recommended that the molecular weight of the polymer be in the range from about 10⁵ to about 10⁷ gram/mol. Furthermore, it is recommended that the active compound/polymer ratio be in the range of about 2:3 to about 9:1, preferably about 3:2 to 9:1, and most preferably about 4:1 to 9:1.

The active compound is preferably dispersed homogeneously within the polymer, wherein the gradual erosion of the polymer in the gastric juice permits extended release of the active compound. Preferred polymers to be used as gastric retention agents are for example synthetic polymers such as Poly(ethylene oxide), polyvinylacetate phthalate, shellac, substituted derivatives thereof and mixtures of any of the foregoing. In another embodiment, cellulose-based polymers may be used for gastric retention. Such polymers are for example hydroxypropyl methylcellulose, hydroxypropylmethylcellulose succinate, cellulose acetate trimellitate, cellulose acetate phthalate, hydroxypropylmethylcellulose phthalate or any other cellulose-based polymers that have been used in the pharmaceutical industry for controlled oral drug delivery systems. Other polymers that possess the ability to swell in water can be used in the present invention.

Examples of such polymers are: poly(hydroxyalkyl methacrylate), poly(electrolyte complexes), poly(vinyl acetate) cross-linked with hydrolysable bonds, water-swellable N-vinyl lactams polysaccharides, natural gum, agar, agrose, sodium alginate, carrageenan, fucoidan, furcellaran, laminaran, hypnea, eucheuma, gum arabic, gum ghatti, gum karaya, gum tragacanth, locust beam gum, arbinoglactan, pectin, amylopectin, gelatin, hydrophilic colloids such as carboxymethyl cellulose gum or alginate gum cross-linked with a polyol such as propylene glycol, and the like. Other polymers that possess the ability to swell in water include hydrophilic hydrogels known as Carbopol, acidic carboxy polymer, Cyanamer, polyacrylamides, polyacrylic acid, polyethylene oxide, starch graft copolymers, acrylate polymer, ester cross-linked polyglucan, and the like.

Other delayed gastric emptying approaches may be used in order to extend the local effect of the active compound in the stomach. These include the use of indigestible polymers or fatty acid salts that change the motility pattern of the stomach to a fed state, thereby decreasing the gastric emptying rate and permitting considerable prolongation of drug release (disclosed for example in Singh and Kim, J. of Controlled Release 63 (2000) 235-259).

The oral dosage forms may be in the form of tablets, capsules, troches, lozenges, aqueous or oily suspensions, dispersible powders or granules, emulsions, multiparticulate formulations, syrups, elixirs, and the like.

Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., antibiotics. For oral application, particularly suitable are tablets, dragees, liquids, drops, suppositories, or capsules, caplets and gelcaps.

The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In connection with the present invention, compounds may be employed, in general, that possess an endoperoxide group that reacts in the presence of ferrous iron to form toxic free radicals. Preferred endoperoxide compounds are set forth hereinabove, although as will be apparent from the present specification that other endoperoxide compounds not specifically mentioned should also be useful in the methods of inhibiting ferrous-dependent bacteria.

H. pylori is a microaerophilic gram-negative bacterium that is associated with multiple gastrointestinal pathologies, such as gastric peptic ulcer, duodenal peptic ulcer, gastritis, duodenitis, non-ulcer dyspepsia and gastric carcinoma. Thus, the active compound of the present invention may be used for prevention and treatment of any pathology associated with H. pylori.

Since in a majority of cases, gastric peptic ulcer is considered to be the result of bacterial infection by H. pylori, the compositions of the present invention may be used for prevention and treatment of any gastrointestinal pathology associated with clinical complaints associated with gastric acid secretion and H. pylori infection, e.g. in patients on nonsteroidal anti-inflammatory drugs (NSAID) therapy (including low dose aspirin), in patients with Non Ulcer Dyspepsia, in patients with symptomatic gastro-esophageal reflux disease (GERD) who need long term PPI treatment, in patients with acute upper gastrointestinal bleeding, and in conditions of stress ulceration. Further, the compositions of the present invention may be used for treating conditions such as Zollinger-Ellison syndrome (ZES), Werner's syndrome, and systemic mastocytosis.

The compositions of the endoperoxide compounds of the invention generally comprise an amount of the endoperoxide compounds sufficient to inhibit growth of the ferrous-dependent bacteria, together with a pharmaceutically acceptable carrier. The compositions are typically administered to a human or other animal subject in an amount to localize a sufficient amount of the endoperoxide compounds at the stomach to facilitate inhibition of the bacterial growth and most preferably, substantial eradication. Any pharmaceutically acceptable carrier may be generally used for this purpose, provided that the carrier does not significantly interfere with the stability or bioavailability of the sesquiterpene compounds of the invention.

The compositions of the invention can be administered in any effective pharmaceutically acceptable form to warm blooded animals, including human and other animal subjects, e.g., oral, suppository, parenteral, or infusable dosage forms, or in any other manner effective to deliver the agents to the target tissue. The route of administration will preferably be designed to optimize delivery and localization of the agents to the target tissue.

Compositions designed for injection may comprise pharmaceutically acceptable sterile aqueous or nonaqueous solutions, suspensions or emulsions. Examples of suitable nonaqueous carriers, diluents, solvents, or vehicles include propylene glycol, polyethylene glycol, vegetable oils, such as olive oil, and injectable organic esters such as ethyl oleate. Such compositions may also comprise adjuvants such as preserving, wetting, emulsifying, and dispensing agents. They may be sterilized, for example, by filtration through a bacteria-retaining filter, or by incorporating sterilizing agents into the compositions. They can also be manufactured in the form of sterile solid compositions that can be dissolved or suspended in sterile water, saline, or other injectable medium prior to administration.

Solid dosage forms for oral administration include capsules, tablets, pills, suppositories, powders, and granules. In solid dosage forms, the compositions may be admixed with at least one inert diluent such as sucrose, lactose, or starch, and may additionally comprise lubricating agents, buffering agents, enteric coatings, and other components well known to those skilled in the art.

The concentrations of the endoperoxide-containing compounds in the formulations to be applied in the practice of the present invention will generally range up to the maximally tolerated dosage, but the concentrations are not critical and may vary widely. For artemisinin and its analogs, however, best results will be obtained using formulations containing the compounds at levels of from about 0.1 to about 100 mg per kilogram of body weight per day, preferably from about 1 to about 90 mg per kilogram of body weight per day, and most preferably from about 1 to about 75 mg per kilogram of body weight per day. The precise amounts employed by the attending physician will vary, of course, depending on the compound, route of administration, and physical condition of the patient and other factors. The daily dosage may be administered as a single dosage or may be divided into multiple doses for administration. The amount of the compound actually administered for treatment will be a therapeutically effective amount, which term is used herein to denote the amount needed to produce a substantial clinical improvement or an amount sufficient to inhibit growth of the bacteria in the subject. Optimal amounts will vary with the method of administration, and will generally be in accordance with the amounts of conventional medicaments administered in the same or a similar form. Oral administration, for instance, may typically be done from once to three times a day.

Combinations of the endoperoxide-containing compound with other antibiotics or a proton pump inhibitor can be administered in a similar manner. Preferred antibiotics are for example: amoxicillin, clarithromycin or other macrolides, metronidazole and related antibiotics, tetracycline, quinolones, rifabutin or furazolidone. Preferred proton pump inhibitor is for example: omeprazole, rabeprazole, lansoprazole, pantoprazole and derivatives or analogues thereof.

The endoperoxide-containing substances are administered both orally and parenterally, alone or in a further combination with pharmaceutically utilizable vehicles. On oral administration, the suitable pharmaceutical vehicles include inert diluents or extenders used for the preparation of tablets, powders, capsules or the like. These pharmaceutical combinations can, if this is desired, contain additional ingredients such as flavorings, binders, corrigents or the like. For example, tablets that contain various corrigents such as sodium citrate, together with various soluble substances such as starch, alginates and certain complex silicates and binders such as polyvinylpyrrolidone, sucrose, gelatin and gum arabic, are used. In addition, lubricants such as magnesium stearate, sodium lauryl sulfate and talc are often suitable for the preparation of tablets. Solid compositions of a similar nature are also used as fillers in filled soft and hard gelatin capsules. Accordingly, the preferred materials include lactose and polyethylene glycols of high molecular weight.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

The following examples are not intended to limit the scope of the invention, but merely to illustrate representative possibilities concerning the present invention.

### Example 1

### Artemisinin Exhibits Strong and Specific Antibacterial Activity Against H. pylori

To test the effect of artemisinin on the growth of H. pylori, freshly prepared bacteria were exposed to various concentrations of artemisinin. Bacteria were grown for fixed incubation times and their growth levels were monitored using a spectrophotometer. The growth of treated bacteria was compared to that of non-treated bacteria. As shown in Table 1, the minimal inhibitory concentration (MIC) of artemisinin for H. pylori is 2.5 μM, suggesting a high antibacterial property for this compound.

To further determine the antibacterial effect of artemisinin, H. pylori maintained in the presence of various concentrations of artemisinin were plated on solid medium and the number of single colonies was monitored. As shown in Table 1, bacteria treated with artemisinin in a concentration equal or higher than 125-250 μM were incapable of forming colony-forming units. These results suggest that 125-250 μM of artemisinin might be considered as a minimal bactericidal concentration (MBC). Thus, artemisinin is highly active against H. pylori. These results could be possibly explained by the high intracellular ferrous iron accumulation present in H. pylori.

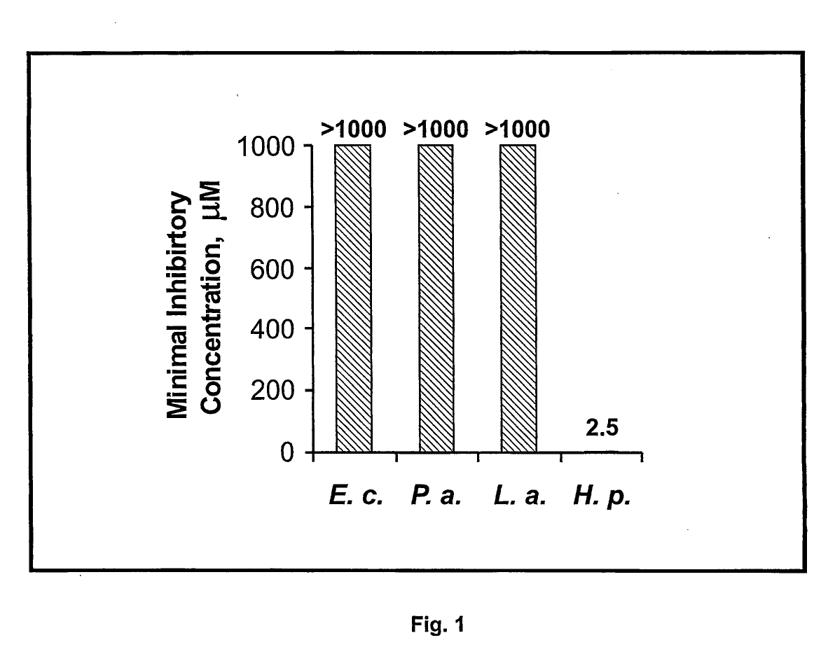

To determine whether the effect of artemisinin is specific to H. pylori, the ability of artemisinin to inhibit the growth of E. coli and P. acnes was tested. In contrary to H. pylori, which is a microaerophilic bacterium, E. coli is an aerobic intestinal bacterium, and P. acnes is an anaerobic dermal bacterium. These bacteria were exposed to various concentrations of artemisinin, and MIC and MBC values were determined. Both E. coli and P. acnes were resistant to milimolar concentrations of artemisinin, suggesting that its inhibitory effect might be limited to ferrous-dependent bacteria, such as, H. pylori (Table 1 and FIG. 1).

The effect of artemisinin on the growth and viability of Lactobacillus acidophilus (L. acidophilus) that is part of the normal flora of the intestine was examined. L. acidophilus bacteria were grown with various concentration of artemisinin and its effect was determined. As shown in Table 1 and FIG. 1, artemisinin did not affect the growth of L. acidophilus at any used concentration below 5 mg/ml. These results indicate that artemisinin utilization will likely not interfere with the normal flora of the intestine.

TABLE 1

| | | |
|---|---|---|
| Bacteria Name | MIC* | MBC** |

| | | | | |
|---|---|---|---|---|
| H. pylori | 2.5 | μM | 125-250 | μM |
| E. coli | >5 | mM | >5 | mM |
| P. acnes | >5 | mM | >5 | mM |
| L. acidophilus | >5 | mM | >5 | mM |

*MIC is a minimal inhibitory concentration.
**MBC is a minimal bactericidal concentration.

### Example 2

### Clarithromycin- and metronidazole-resistant strains of H. pylori are sensitive to artemisinin

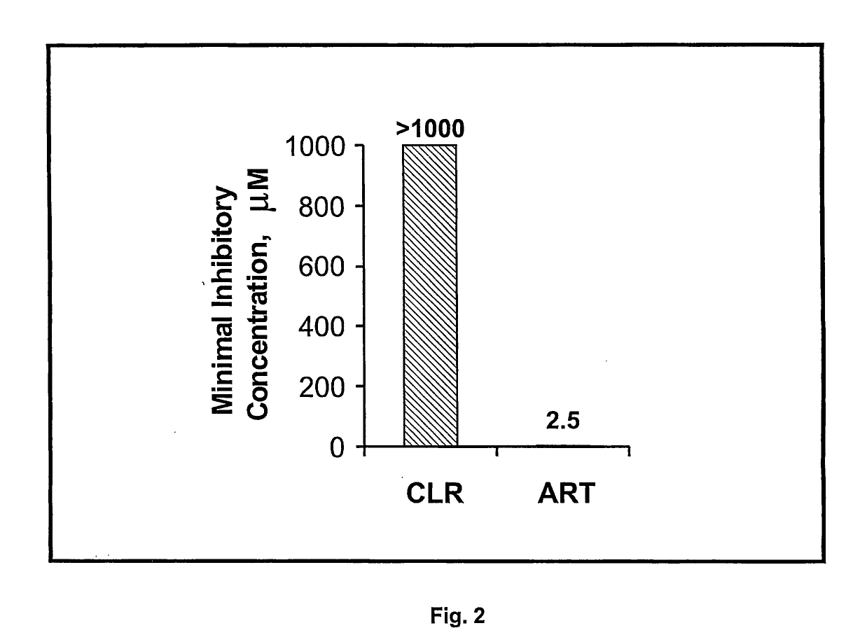

Unsuccessful therapy in patients infected with H. pylori is frequently correlated to clarithromycin and metronidazole resistance. To evaluate the potential of using artemisinin against H. pylori isolates with antibiotic resistance, the effect of artemisinin was tested with bacteria strains that exhibit resistance to clarithromycin and metronidazole. H. pylori exhibiting resistance to clarithromycin (CLR) and metronoidazole were grown in the presence of 1 mM of CLR or 2.5-10 μM of artemisinin. Bacterial cultures were incubated in micro-aerophilic conditions. The effect of clarithromycin or artemisinin on bacterial growth was tested using a spectrophotometer after 3 days. As demonstrated in FIG. 2, the tested resistant strains possess significant sensitivity to artemisinin. This finding indicates that artemisinin might be considered as a good candidate for treating patients infected with resistant strains of H. pylori.

### Example 3

### Artemisinin and Omeprazole Inhibit Synergistically the Growth of H. pylori

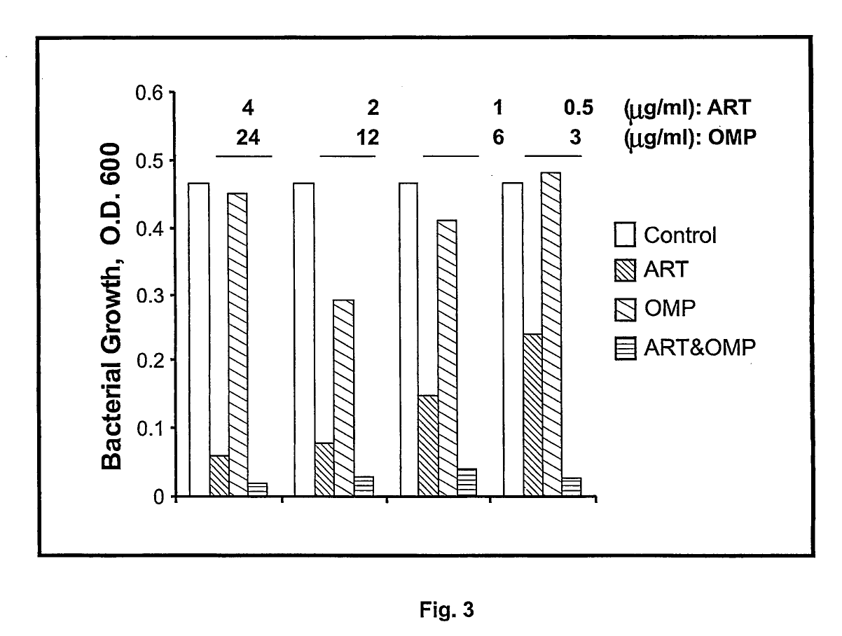

In order to explore the possible synergism between artemisinin and a PPI, the growth of bacteria in the presence of artemisinin, omeprazole or both was examined. H. pylori bacteria were grown in the presence of 3-24 μg/ml omeprazole or 0.5-4 μg/ml of artemisinin. Bacterial cultures were incubated in micro-aerophilic conditions. The combined effect of artemisinin and omeprazole on bacterial growth was tested using a spectrophotometer after 3 days. As demonstrated in FIG. 3, it is sufficient to administer 3 μg/ml omeprazole in combination with 0.5 μg/ml artemisinin in order to obtain a dramatic decrease of bacterial growth. These results suggest that the combination of omeprazole and artemisinin administered in concentrations in which the drugs are not effective alone possesses a synergistic inhibitory effect on the growth of H. pylori.

### Example 4

### Artemisinin Derivatives Inhibit the Growth of H. pylori

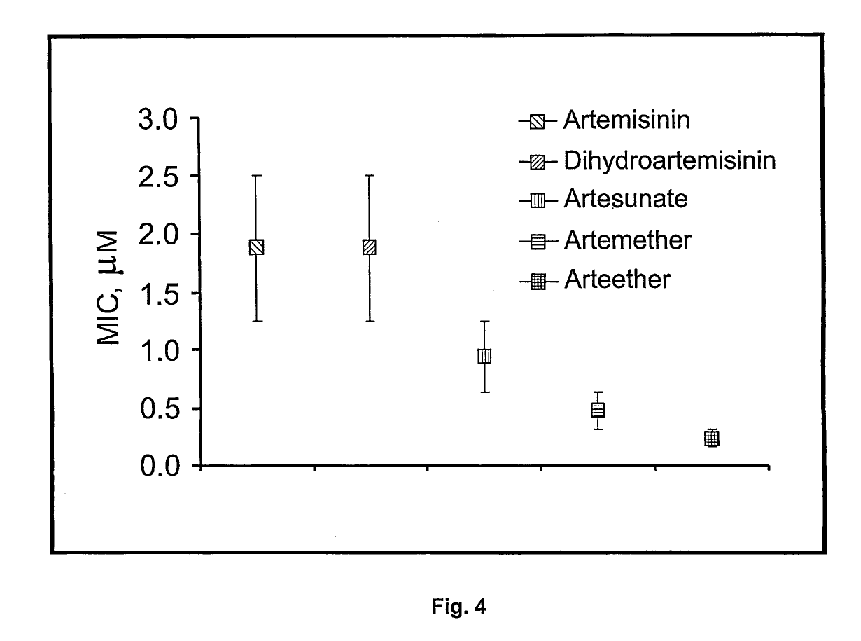

To test the effectivity of artemisinin derivatives on the growth of H. pylori, freshly prepared bacteria were exposed to various concentrations of artemisinin derivatives. Bacteria were grown for fixed incubation times and their growth levels were monitored using a spectrophotometer. The growth of treated bacteria was compared to that of non-treated bacteria. As shown in FIG. 4, the minimal inhibitory concentration (MIC) values of artemisinin derivatives for H. pylori are as following: artemisinin and dihydroartemisinin=1.25-2.5 μM, artemether=0.3-0.6 μM, arteether=0.15-0.3 μM. Thus, all artemisinin derivatives examined possess anti-H. pylori activity.

### Example 5

### Prolonged Exposure of Artesunate to H. pylori Bacterial Cultures Results in Irreversible Bacterial Eradication

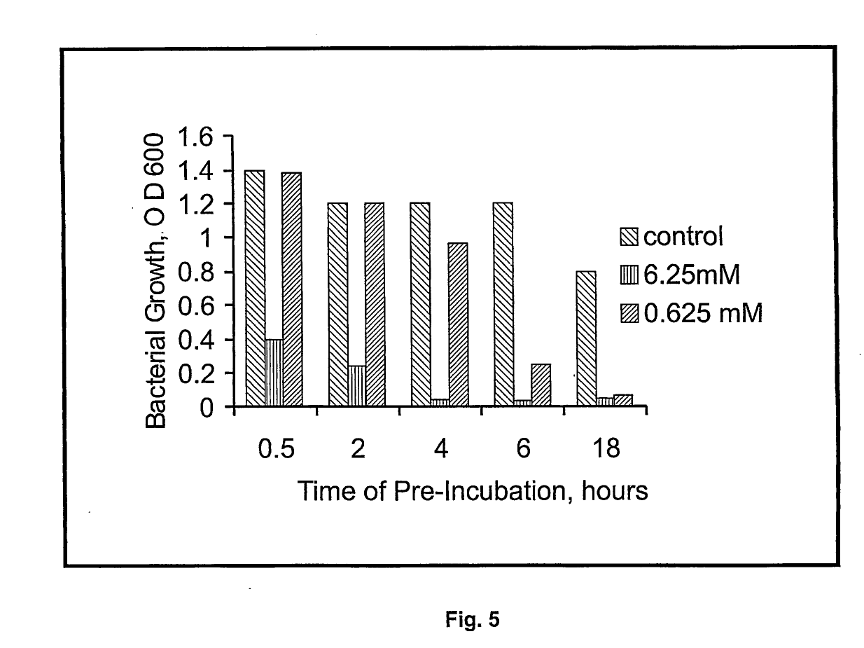

Artesunate (0.625 or 6.25 mM) was added to bacterial cultures (0.2 O.D.₆₀₀) for various time points (0.5, 1, 2, 4, 6, and 18 hours). Artesunate was then washed out from the cultures by precipitation of the bacteria and washing in PBS, after which bacteria were re-suspended in a fresh artesunate-free medium. Bacteria were maintained in artesunate-free medium for additional 36 hours and bacterial growth was then determined by spectrophotometer. The results demonstrated in FIG. 5 indicate that prolonged exposure of H. pylori bacterial cultures to artesunate results in irreversible bacterial eradication.

### Example 6

### Artesunate Preserves its Anti-Helicobacter Activity after Long Incubation in Low pH Conditions

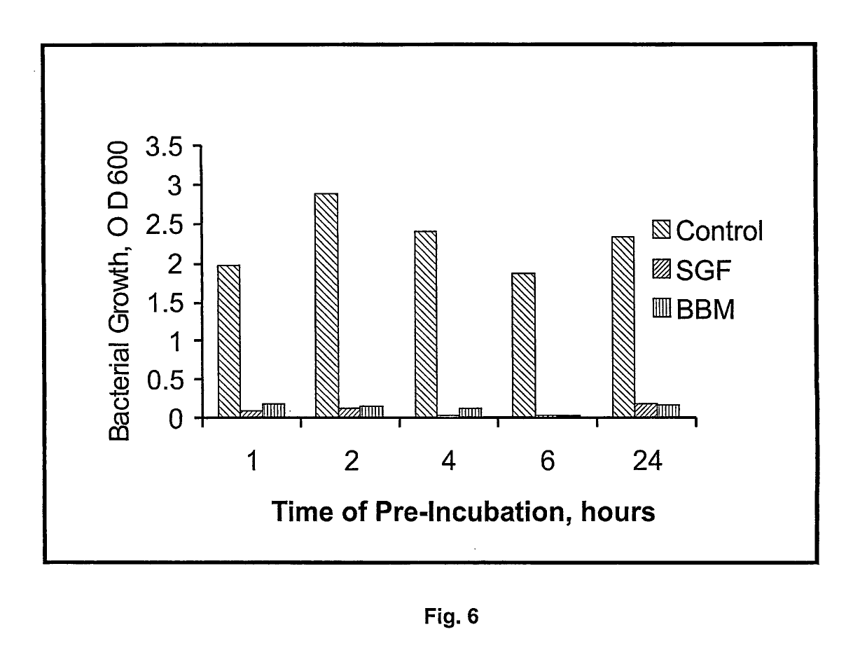

Artesunate (1 mg/ml) was pre-incubated in simulated gastric fluid (SGF, pH 1.2) or in bacterial broth medium (BBM, natural pH) at 37° C. for various time periods (1, 2, 4, 6, and 24 hours). After the pre-incubation, bacterial cultures (0.01 O.D) were exposed for 36 h to culture medium containing the pre-incubated artesunate. The effect of artesunate on bacterial growth was then determined by spectrophotometer. The results demonstrated in FIG. 6 indicate that the activity of Artesunate is preserved even following 24 h pre-incubation in acidic conditions.

### Example 7

### Artesunate Efficiently Reduces the Number of Colony Forming Units in H. pylori-Infected Mice Treated with Artesunate Versus Placebo

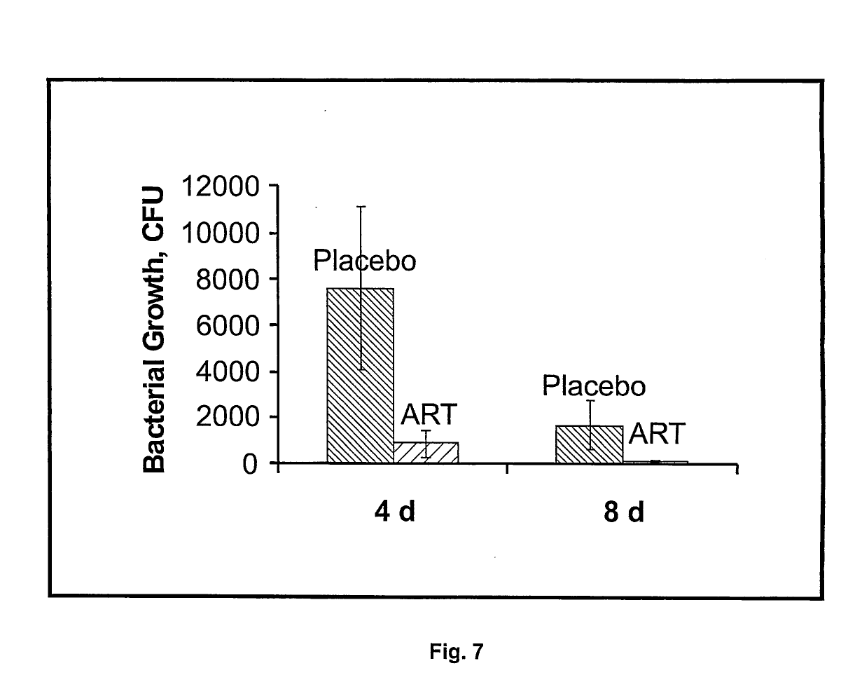

The effect of artesunate against H. pylori infection in vivo was tested in H. pylori-infected mice. Mice were infected by inoculation (×3/day) with suspension of 10⁹ H. pylori bacterial strain SS1. Two weeks following the infection, mice were treated orally with 50 mg/kg artesunate 3 times/day for 8 days. The level of bacterial infection was determined by counting the number of colony forming units derived from the homogenized stomach on day 4 and 8 of the treatment. As shown in FIG. 7, artesunate efficiently reduced the number of colony forming units in mice treated with artesunate versus placebo suggesting that the artesunate is capable of eliminating H. pylori in vivo.

### Example 8

### Hard Gelatin Capsules Comprising Artesunate in Mini-Tabs, Enteric-Coated Omeprazole Beads, and Calcium Carbonate

Hard gelatin capsules are formulated as a single dosage form comprising mixed population of particles. Each capsule contains the following ingredients:
40 mg omeprazole as enteric-coated beads
250 mg artesunate granules
550 mg calcium carbonate (CaCO₃)
hydroxypropyl methylcellulose (HPMC) K100M
Polyox WSR N60

Artesunate is granulated in combination with HPMC, Polyox and CaCO₃ and compressed into mini-tabs. The mini-tabs possess the ability of fast swelling upon contact with the gastric juice of the stomach, thereby enabling gastric retention and local activity of artesunate within the gastric mucosa. The release of artesunate and CaCO₃ into the stomach is controlled by the erosion rate of the polymeric matrix of the swelled mini-tabs. The artesunate mini-tabs together with the enteric-coated omeprazole beads are packed into size 0 hard gelatin capsules in an amount corresponding to 40 mg omeprazole, 250 mg artesunate and 550 mg calcium carbonate per capsule.

### Example 9

### Multi Particulate Capsules Containing Enteric-Coated Omeprazole and Artesunate Beads

This example illustrates the steps involved in manufacturing multi particulate hard gelatin capsules. Capsules are formulated as a single dosage form comprising mixed population of particles: artesunate beads and enteric-coated omeprazole beads. Each capsule contains the following ingredients:
40 mg enteric-coated omeprazole beads
250 mg artesunate granules

### Example 10

### Enteric-Coated Tablets Comprising Artesunate Powder and Omeprazole Powder

Pressed tablets are formulated as a single dosage form containing the following ingredients:
40 mg omeprazole powder
250 mg artesunate powder

Pressed tablets are prepared by mixing and pressing 250 mg artesunate powder and 40 mg of omeprazole powder The final tablet is coated with enteric-coating to permit systemic absorption of the active ingredients in the intestine. In another example, the active ingredients are compressed into double-layered tablet wherein the first layer comprises 250 mg artesunate and the second layer comprises 40 mg of omeprazole powder. The final tablet is then coated with enteric-coating.

The compressed tablet may include one or more of the following excipients: lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers.

Any and all publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

It will be appreciated by a person skilled in the art that the present invention is not limited by what has been particularly shown and described hereinabove. Rather, the scope of the invention is defined by the claims that follow.

What is claimed is:

1. A method of inhibiting the growth of a ferrous-dependent bacteria in a subject containing same, comprising administering to the subject in need thereof an endoperoxide-containing compound, the compound being in an amount sufficient to inhibit the growth of the ferrous-dependent bacteria.

2. The method of claim 1, wherein the endoperoxide-containing compound is selected from the group consisting of: sesquiterpene lactones and alcohols, carbonates, esters, ethers sulfonates and pharmaceutically acceptable salts thereof, trioxolanes, byciclo endoperoxides, trioxanes, tetraoxanes, terpenes, and substituted terpenes.

3. The method of claim 2, wherein the endoperoxide-containing compound is according to formula (I): 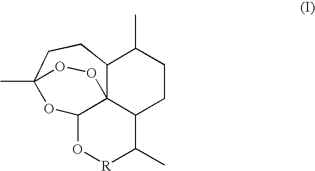
wherein R is —CO— or R is —CR₁—;
R₁ is hydrogen, hydroxyl, alkyl, —OR₂, —COR₂, —COR₂, —COOR₂, —CO(CH₂)ₙ, —COOH, or —SOOR₂;
R₂ is alkyl or aryl; and n is 1 to 6.

4. The method of claim 3, wherein the endoperoxide-containing compound is a sesquiterpene selected from the group consisting of: artemisinin, dihydroartemisinin, artemether, arteether, arteflene, artesunate, dihydroxydihydroartemisinin, artelinic acid, and dihydroartemisinin propyl carbonate.

5. The method of claim 1, further comprising administering to the subject a therapeutically effective amount of at least one active agent selected from the group consisting of: an antibiotic agent, an inhibitor of gastric acid secretion, a proton pump inhibitor (PPI), a reversible proton pump inhibitor, an H2 blocker, a bismuth-containing compound, a cytoprotectant, a prostaglandin analogue, and iron.

6. The method of claim 5, wherein the PPI is selected from the group consisting of: rabeprazole, omeprazole, esomeprazole, lansoprazole, pantoprazole, leminoprazole, tenatoprazole, single enantiomers thereof, alkaline salts thereof, and mixtures thereof.

7. The method of claim 5, wherein the antibiotic agent is selected from the group consisting of: amoxicillin, a macrolide, metronidazole, tetracycline, quinolones, rifabutin, and furazolidone.

8. The method of claim 1, wherein the ferrous-dependent bacteria is Helicobacter pylori.

9. A method of treating or preventing Helicobacter sp-related disorder in a subject in need of such treatment, which comprises administering to the subject an endoperoxide-containing compound in an amount sufficient to treat or prevent the Helicobacter sp-related disorder.

10. The method of claim 9, wherein the endoperoxide-containing compound is selected from the group consisting of: sesquiterpene lactones and alcohols, carbonates, esters, ethers sulfonates and pharmaceutically acceptable salts thereof, trioxolanes, byciclo endoperoxides, trioxanes, tetraoxanes, terpenes, and substituted terpenes.

11. The method of claim 10, wherein the endoperoxide-containing compound is according to formula (I): 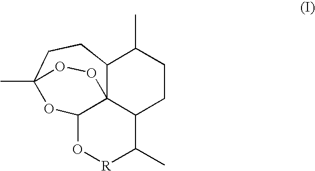
wherein R is —CO— or R is —CR₁—;
R₁ is hydrogen, hydroxyl, alkyl, —OR₂, —COR₂, —COR₂, —COOR₂, —CO(CH₂)ₙ, —COOH, or —SOOR₂;
R₂ is alkyl or aryl; and n is 1 to 6.

12. The method of claim 11, wherein the endoperoxide-containing compound is a sesquiterpene selected from the group consisting of: artemisinin, dihydroartemisinin, artemether, arteether, arteflene, artesunate, dihydroxydihydroartemisinin, artelinic acid, and dihydroartemisinin propyl carbonate.

13. The method of claim 9, further comprising administering to the subject a therapeutically effective amount of at least one active agent selected from the group consisting of: an antibiotic agent, an inhibitor of gastric acid secretion, a proton pump inhibitor (PPI), a reversible proton pump inhibitor, an H2 blocker, a bismuth-containing compound, a mucoadhesive agent, a prostaglandin analogue, and iron.

14. The method of claim 13, wherein the PPI is selected from the group consisting of: rabeprazole, omeprazole, esomeprazole, lansoprazole, pantoprazole, leminoprazole, tenatoprazole, single enantiomers thereof, alkaline salts thereof, and mixtures thereof.

15. The method of claim 13, wherein the antibiotic agent is selected from the group consisting of: amoxicillin, a macrolide, metronidazole, tetracycline, quinolones, rifabutin, and furazolidone.

16. The method of claim 9, wherein the Helicobacter sp-associated disorder is a Helicobacter sp-associated gastrointestinal disorder.

17. The method of claim 16, wherein the Helicobacter sp-associated gastrointestinal disorder is selected from: gastric peptic ulcer, duodenal peptic ulcer, gastritis, duodenitis, non-ulcer dyspepsia, MALTOMA, intestinal metaplasia of the stomach, and gastric carcinoma.

18. The method of claim 16, wherein the gastrointestinal disorder is caused by Helicobacter pylori.

19. The method of claim 18, wherein the Helicobacter pylori strain is resistant to clarithromycin, or metronidazole.

20. The method of claim 9, wherein the endoperoxide-containing compound is administered by intravenous, parenteral, or oral means.

21. The method of claim 18, wherein the endoperoxide-containing compound substantially eradicates the bacteria.

22. A pharmaceutical composition for treating or preventing Helicobacter sp-related gastrointestinal disorders comprising:
(a) a pharmaceutically effective amount of a compound according to formula (I): 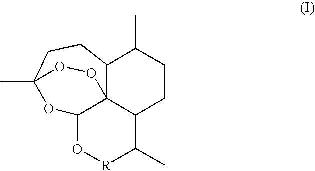
wherein R is —O— or R is —CR₁—;
R₁ is hydrogen, hydroxyl, alkyl, OR₂, —COR₂, —COR₂, —COOR₂ —CO(CH₂)ₙ, —COOH, or —SOOR₂;
R₂ is alkyl or aryl; and n is 1 to 6; and
(b) one or more additional active agents selected from the group consisting of: an antibiotic agent, an inhibitor of gastric acid secretion, a proton pump inhibitor (PPI), a reversible proton pump inhibitor, an H2 blocker, a bismuth-containing compound, a mucoadhesive agent, a prostaglandin analogue, and an anti-inflammatory agent.

23. The composition of claim 22, wherein the Helicobacter sp-related gastrointestinal disorder is a Helicobacter pylori-related gastrointestinal disorder.

24. The composition of claim 22, further comprising at least one ingredient selected from the group consisting of: iron, one or more mucolytic agents, one or more gastric retentive agents, cyclodextrin, and one or more alkaline agents.

25. The composition of claim 22, wherein the oral composition is in the form of a tablet, a capsule, solution, powder for suspension, dispersion, or emulsion.

26. The composition of claim 22, wherein the compound is a sesquiterpene selected from the group consisting of: artemisinin, dihydroartemisinin, artemether, arteether, arteflene, artesunate, dihydroxydihydroartemisinin, artelinic acid, and dihydroartemisinin propyl carbonate.

27. The composition of claim 22, wherein the PPI is selected from the group consisting of: rabeprazole, omeprazole, esomeprazole, lansoprazole, pantoprazole, leminoprazole, tenatoprazole, single enantiomers thereof, alkaline salts thereof, and mixtures thereof.

28. The composition of claim 22, wherein the antibiotic agent is selected from the group consisting of: amoxicillin, a macrolide, metronidazole, tetracycline, quinolones, rifabutin, and furazolidone.

29. The composition of claim 22 wherein the compound according to formula (I) is artemisinin or artesunate, and the PPI is omeprazole.

30. The composition of claim 22, wherein the compound according to formula (I) and the active agent for treating Helicobacter sp-related gastrointestinal disorders are in a ratio of from about 50:1 to about 1:100.

31. The composition of claim 22, wherein the Helicobacter sp-associated gastrointestinal disorder to be treated or prevented is selected from the group consisting of: gastric peptic ulcer, duodenal peptic ulcer, gastritis, duodenitis, non-ulcer dyspepsia, MALTOMA, intestinal metaplasia of the stomach, and gastric carcinoma.
